(19) United States
(12) Patent Application Publication
(10) Pub. No.: US 2024/0325033 A1
(43) Pub. Date: Oct. 3, 2024

(54) BONE FRAGMENT MANIPULATION DEVICE AND METHODS OF PRODUCTION AND USE THEREOF

(71) Applicant: DePuy Synthes Products, Inc., Raynham, MA (US)

(72) Inventors: Emily BROUDE, West Chester, PA (US); Jordan CONLEY, West Chester, PA (US)

(21) Appl. No.: 18/191,563

(22) Filed: Mar. 28, 2023

(51) Int. Cl. A61B 17/16

(52) U.S. Cl. CPC A61B 17/1697; A61B 2017/564

(57) ABSTRACT

Bone fragment manipulation devices for use at surgical sites are disclosed. These devices include a handle disposed between a first tool component and a second tool component. The second tool component includes an opening for receiving a k-wire. Systems and kits including these devices, as well as methods of production and use thereof, are also disclosed.

### CROSS REFERENCE TO RELATED APPLICATIONS/INCORPORATION BY REFERENCE STATEMENT

Not Applicable.

### STATEMENT REGARDING FEDERALLY SPONSORED RESEARCH OR DEVELOPMENT

Not Applicable.

### BACKGROUND

In trauma surgery, a surgeon seeks to manipulate small fragments of bone directly or via a Kirschner wire (i.e., k-wire). Bone fragments can be manipulated and held in place with pushers, dental hooks, and plungers, for example. K-wires may then be used to hold bone fragments together.

The manipulation of bone fragments, especially in a die punch fracture, requires precision, accuracy and specific force. The manipulation of bone fragments during trauma also requires specific timing. Pushers, dental hooks and plungers are limited in the amount of time each is able to hold a fragment of bone in a particular position. Having a surgeon change hands or instruments during the procedure may result in a potential loss of the bone fragment or displacement of a K-wire. While a conventional pusher may be able to provide force to manipulate a fragment of bone, the subsequent step of positioning a k-wire requires the surgeon to change hands and instruments that may result in inadvertent repositioning of the pusher.

The space allowed within a surgical field is also limited. Allocating multiple devices or multiple people to manipulate bone fragments and hold bone fragments hinders use of this space.

Therefore, there is a need in the art for new and improved devices and methods of producing and using the same in manipulating bone fragments during trauma surgert. It is to such devices, as well as methods of producing and using same, that the present disclosure is directed.

### BRIEF DESCRIPTION OF THE DRAWINGS

FIG. 1 is a perspective view of a bone fragment manipulation device in accordance with the present disclosure.

FIGS. 2A-2C are side views of exemplary handles for use in the bone fragment manipulation device of FIG. 1.

FIGS. 3A-3D are side views of exemplary first tool components for use in the bone fragment manipulation device of FIG. 1.

FIG. 4A is a side view of an exemplary second tool component for use in the bone fragment manipulation device of FIG. 1.

FIG. 4B is a perspective view of an exemplary bone fragment manipulation device in accordance with the present disclosure having mirroring congruent first and second tool components in accordance with the present disclosure.

FIG. 4C is a perspective view of another exemplary bone fragment manipulation device having first, second and third tool components in accordance with the present disclosure.

FIG. 5A is a cross-sectional view of an exemplary guiding member in accordance with the present disclosure.

FIG. 5B is a cross-sectional view of the exemplary guiding member of FIG. 5A positioned within a distal end of the bone fragment manipulation device of FIG. 1.

FIG. 6A is a perspective view of another exemplary guiding member in accordance with the present disclosure.

FIG. 6B is a cross-sectional view of the exemplary guiding member of FIG. 6A.

FIG. 6C is a cross-sectional view of the exemplary guiding member of FIG. 6A positioned within a distal end of an exemplary bone fragment manipulation device in accordance with the present disclosure.

FIG. 7 is a perspective view of one embodiment of the presently disclosed and claimed inventive concept(s).

FIG. 8 is a top view of one embodiment of the presently disclosed and claimed inventive concept(s).

FIG. 9 is a left side view of one embodiment of the presently disclosed and claimed inventive concept(s).

FIG. 10 is a bottom view of one embodiment of the presently disclosed and claimed inventive concept(s).

FIG. 11 is right side view of one embodiment of the presently disclosed and claimed inventive concept(s).

FIG. 12 is a front view of one embodiment of the presently disclosed and claimed inventive concept(s).

FIG. 13 is a rear view of one embodiment of the presently disclosed and claimed inventive concept(s).

FIG. 14 is a perspective view of an alternative embodiment of the presently disclosed and claimed inventive concept(s).

FIG. 15 is a right side view of an alternative embodiment of the presently disclosed and claimed inventive concept(s).

FIG. 16 is a left side view of an alternative embodiment of the presently disclosed and claimed inventive concept(s).

FIG. 17 is a front view of an alternative embodiment of the presently disclosed and claimed inventive concept(s).

FIG. 18 is rear view of an alternative embodiment of the presently disclosed and claimed inventive concept(s).

FIG. 19 is a bottom view of an alternative embodiment of the presently disclosed and claimed inventive concept(s).

FIG. 20 is a top view of an alternative embodiment of the presently disclosed and claimed inventive concept(s).

Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present disclosure.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary and not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the medical procedures and techniques of trauma surgery described herein are those well-known and commonly used in the art. Standard techniques are used for diagnostic and therapeutic applications.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, compositions, kits, and/or methods disclosed herein can be made and executed without undue experimentation in light of the present disclosure. While the articles, compositions, kits, and/or methods have been described in terms of particular embodiments, it will be apparent to those of skill in the art that variations may be applied to the articles, compositions, kits, and/or methods and in the steps or in the sequence of steps of the methods described herein without departing from the concept, spirit, and scope of the present disclosure. All such similar substitutions and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a device” may refer to one or more devices, two or more devices, three or more devices, four or more devices, or greater numbers of devices. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and, unless explicitly stated otherwise, is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the term “patient” or “subject” is meant to include all organisms, whether alive or dead, including any species having soft tissues and bones. For example, a system according to the inventive concepts disclosed herein may be applied to the skin of a living human, horse, cow, sheep, cat, dog, and the like.

The present disclosure is directed to a bone fragment manipulation device for use in surgery to hold and manipulate bone fragments (e.g., die punch fracture) of a patient. The bone fragment manipulation device includes a central handle with two curved ends having functional tools thereon. Curved ends may aid manipulation of the device in a range of directions relative an incision. One end of the device may include a ball and spike used to hold and manipulate bone fragments without damaging the bone. An opposing end of the device may include a cylindrical tamp used to elevate bone fragments, (e.g., die punch fragments). The opposing end may also be cannulated to accept a Kirschner wire (i.e., k-wire). The surgeon is able to use the handle as a joystick to control a bone fragment with the k-wire, ball and spike, and/or tamp. As such, the surgeon is able to use a single hand on the handle of the device to manipulate multiple tools on a single instrument. The present disclosure is also directed to assemblies, systems, and kits that include the bone fragment manipulation device, as well as methods of producing and using same, as described in greater detail herein below.

Certain non-limiting embodiments of the present disclosure are directed to a bone fragment manipulation device for use at a surgical site. The device includes a first tool component having a first elongated member extending from a proximal end to a distal end. The distal end of the first tool component may include a spike, a ball and spike, a tamp, an elevator, a double ball spike, an awl, or the like.

The device also includes a second tool component having a second elongated member extending from a proximal end to a distal end and having an outer surface defining an opening. The opening is configured to receive a k-wire. That is, the opening has a diameter sufficient to allow the k-wire to pass therethrough. As such, the diameter of the opening may be dependent on the particular k-wire being used therewith. Certain non-limiting embodiments of the present disclosure are directed to an assembly that includes a bone fragment manipulation device as described or contemplated herein, and one or more k-wires in combination with the bone fragment manipulation device. Any k-wire known in the art or otherwise contemplated herein may be utilized in accordance with the present disclosure.

The distal end of the second tool component may also include a spike, a ball and spike, a tamp, an elevator, a double ball spike, an awl, or the like. The k-wire may be threaded through the distal end of the second tool component. For example, the tamp may include a serrated edge having multiple projections and a cavity therethrough allowing for the k-wire to be threaded therethrough.

A handle is disposed between the first tool component and the second tool component. The handle allows sufficient friction to exist between the hand or fingers of a surgeon and the handle so that the surgeon may be able to operate the assembly with a single hand. An outer peripheral surface of the handle may have protrusions and recesses that aid in creating friction. The handle allows the surgeon to apply force to the first tool component, the second tool component, or the k-wire to manipulate and move bone fragments at a surgical site. The outer peripheral surface of the handle may also have indicators or marks to aid in placement of a surgeon's hand or fingers.

The first tool component includes the first elongated member extending from the proximal end to the distal end. The first elongated member and the second elongated member, however, may be provided with any shape, configuration, and/or dimensions that allow for the bone fragment manipulation device to function in accordance with the present disclosure. For example, in certain non-limiting embodiments, the first elongated member can extend from the handle at a first angle and can be arched to symmetrically curve from the handle to the distal end. Similarly, the second elongated member of the second tool component can extend from the handle at a second angle and can be arched to symmetrically curve from the handle to the distal end of the second elongated member. The first angle and the second angle can be approximate congruent angles. The first angle and the second angle can be approximate alternate angles. In other non-limiting embodiments, either the first elongated member and/or the second elongated member may be substantially straight. That is, the first elongated member may be substantially straight from the proximal end to the distal end.

In certain non-limiting embodiments, the bone fragment manipulation device may also include a guiding member configured to be fixedly retained within the cavity of the second elongated member. The guiding member includes a sidewall extending from a first end to a second end and defining a first cavity. The first cavity is sized and shaped to retain the k-wire. The guiding member may also include a plurality of flanges positioned on the second end. The flanges are movable from a resting position to an expanded position to retain the k-wire within the first cavity.

In certain non-limiting embodiments, the guiding member may include a connector portion and a shaft. The plurality of flanges are positioned on the connector portion. The connector portion also includes a plurality of protrusions on an exterior surface to engage with recesses on an inner surface of the elongated member. Application of force to the guiding member in the direction towards the distal end of the second tool component compresses the plurality of flanges on the k-wire within the connector portion of the guiding member retaining the k-wire within the guiding member. Application of force to the guiding member in the direction towards the proximal end of the second tool component decreases compressive force of the plurality of flanges on the k-wire within the connector portion of the guiding member releasing the k-wire within the guiding member.

In certain non-limiting embodiments, the bone manipulation device may include a third tool component. The third tool component may be provided with any shape, configuration and/or dimensions that allow the bone fragment manipulation device to function in accordance with the present disclosure. The third tool component may be positioned on the same side of the handle as the second tool component. The third tool component is similar to the second tool component and has a third elongated member extending from the handle. The third elongated member has an outer surface defining an opening and configured to receive a second k-wire. A distal end of the third elongated member may include a spike, a ball and spike, a tamp, an elevator, a double ball spike, an awl, or the like.

Certain non-limiting embodiments of the present disclosure are directed to a kit that contains one or more bone fragment manipulation device described or otherwise contemplated herein. The kit may include a single bone fragment manipulation device or a plurality of bone fragment manipulation devices. When a plurality of bone fragment manipulation devices are present, each device may have the same size and shape, or the devices may have two or more different sizes and/or shapes. For example, the positioning and shape of the elongated members may be different in one or more of the devices.

In certain non-limiting embodiments, the kit may further include one or more of any of the assembly or system components described herein. Non-limiting example of these components include at least elongated members, handles, and at least one k-wire.

In certain non-limiting embodiments, the kit may further include one or more instruments that are utilized in the formation and/or placement of any of the assemblies and/or systems described herein. For example, the kit may further include one or more instruments that are utilized at the surgical site to aid in placement of the k-wire(s).

Different components may be present in the kit in any manner disclosed herein or otherwise known in the art. For example (but not by way of limitation), each of the components may be disposed in separate compartments, or two or more of the same or different components may be disposed in the same compartment. In addition, two or more of the components may be associated with one another prior to disposal within the kit. That is, a k-wire may be positioned within a guiding member prior to disposal within a kit.

In addition, the kit can further include a set of written instructions explaining how to use one or more components of the kit. A kit of this nature can be used in any of the methods, described or otherwise contemplated herein.

Certain non-limiting embodiments of the present disclosure are directed to a method of treating a subject, in which any of the assemblies described or otherwise contemplated herein is positioned at a surgical site of the subject. In certain non-limiting embodiments, the surgical site includes a bone fracture (e.g., die punch fracture).

Certain non-limiting embodiments of the present disclosure are direct to a method of treating a subject. In the method, the bone manipulation device is inserted into an incision in skin of the subject and directed to a surgical site. The first tool component of the device is positioned at the surgical site. Force is applied via the handle to allow the spike to manipulate bone fragments at the surgical site. The handle is then rotated such that the second tool component is positioned at the surgical site. Force is applied to the handle such that the tamp is used to hold bone fragments at the surgical site. A k-wire is introduced into the opening in the second tool component. In certain non-limiting embodiments, force is applied to the handle to manipulate bone fragments via the k-wire.

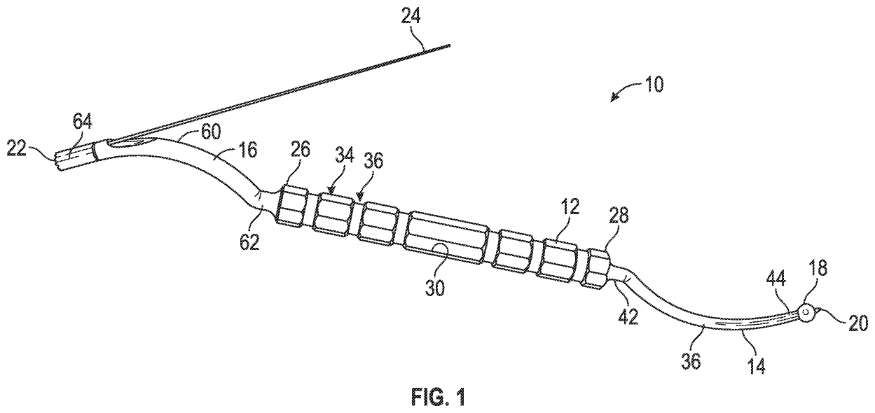

Turning now to the Drawings, FIG. 1 illustrates a bone fragment manipulation device or assembly. The assembly 10 comprises a handle 12 disposed between a first tool component 14 and a second tool component 16. The first tool component 14 includes a ball 18 and spike 20 configured to engage, hold and/or manipulate a fragment of bone. The second tool component 16 includes a tamp 22 configured to engage, hold and/or manipulate a fragment of bone. The first tool component and/or the second tool component 16 may be cannulated to receive a k-wire 24. For example, in FIG. 1, the second tool component 16 is cannulated to receive a k-wire 24. The assembly 10 allows the surgeon to use the handle 12 as a joystick to control a bone fragment with the first tool component 14, the second tool component 16 or the k-wire 24.

The handle 12 is disposed between the first tool component 14 and the second tool component 16 of the assembly 10. The handle 12 may be provided with any structural features that allow sufficient friction to exist between a hand of a surgeon and the handle 12. In addition, the handle 12 may be formed such that a surgeon may be able to operate the assembly 10 with a single hand.

The handle 12 includes a first end 26, a second end 28, and an outer peripheral surface 30 extending between the first end 26 and the second end 28. The structure of the handle 12 shown in FIG. 1 is for purposes of illustration only. In particular, the cylindrical shape of the handle 12 is solely for purposes of illustration only and should not be considered limiting; the handle 12 may assume any shape (including, but not limited to, cube, cuboid, conical, or frustoconical shapes, any combination of one or more shapes, etc.) so long as the shape will allow the handle 12 to function in accordance with the present disclosure.

Diameter of the handle 12 may be formed to allow a hand of a surgeon to supply force to the first tool component 14 or the second tool component 16 of the assembly 10 in accordance with the present disclosure. The length of the handle 12 may allow for a hand of a surgeon to have complete contact with a full length of the handle 12. Alternatively, the length of the handle 12 may allow for multiple fingers of the surgeon to have contact on the length of the handle 12.

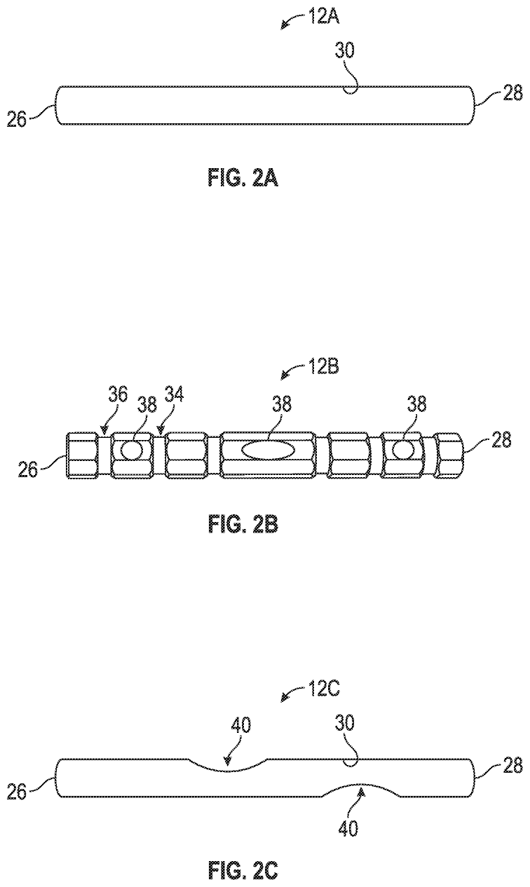

The outer peripheral surface 30 of the handle 12 may be formed of non-slip and non-conductive material. Texture of handle 12 may include a high-friction or etched outer peripheral surface 30. As shown in FIG. 1, the outer peripheral surface 30 of the handle may include recesses 32 and protrusions 34. Patterns formed by the recesses 32 and protrusions 34 may supply additional friction to exist between a hand of a surgeon and the handle 12. Such patterns may assume protrusions 34 or recesses 32 in any shape, size and configuration so long as the handle is capable of allowing the surgeon to provide force and manipulate the first tool component 14 or the second tool component 16 of the assembly 10 in accordance with the present disclosure. Alternatively, the outer peripheral surface 30 may be a uniform surface free from protrusions 34 or recesses 32, as shown in handle 12a of FIG. 2A.

Referring to FIGS. 2B-2C, the outer peripheral surface 30 of the handle 12 may include one or more guides for placement of the hand or fingers of the surgeon. For example, protrusions 34 or recesses 32 may additionally include a mark 38 or indicator directing placement of the hand or finger of the surgeon on the protrusion 34 or in the recesses 32 of the handle 12. Alternatively, one or more indentions 40 may be formed within the outer peripheral surface 30 of the handle 12 for placement of the hand or fingers of the surgeon on the handle 12. Indentions 40 may also be formed on protrusions 34 or in recesses 32 of the handle 12.

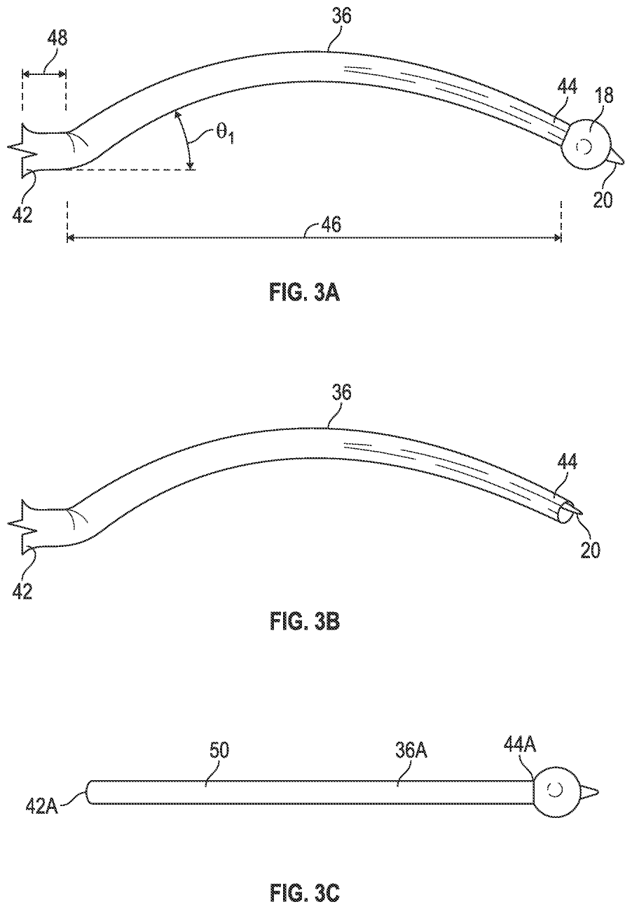

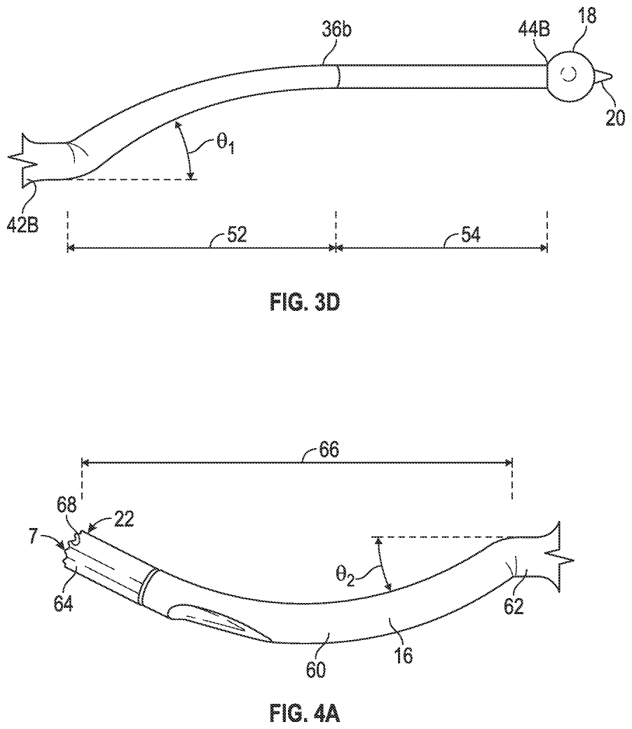

Referring to FIGS. 1 and 3A-3C, the first tool component 14 of the assembly 10 includes an elongated member 36 extending from a proximal end 42 to a distal end 44. The elongated member 36 may be formed as a solid rod or a hollow tube extending from the proximal end 42 to the distal end 44. The elongated member 36 is shown in FIGS. 1 and 3A having arched portion 46. The arched portion 46 may extend from the handle 12 at an angle of extension 01 relative to the handle 12 of the assembly 10. The arched portion 46 may symmetrically curve from the proximal end 42 to the distal end 44 of the elongated member 36. The angle of extension 01 of the arched portion 46 from the handle 12 may be configured to allow for a surgeon to apply force in a range of directions relative an incision.

The proximal end 42 of the first tool component 14 of the assembly 10 is attached to or integrally formed with the handle 12 of the assembly 10. A portion 48 of the proximal end 42 of the first tool component 14 may extend from the handle 12 of the assembly 10 as shown in FIG. 1. The portion 48 of the proximal end 42 may axially aligned with the handle 12.

The distal end 44 of the first tool component 14 of the assembly 10 includes a functional tool configured to engage, hold and/or manipulate a fragment of bone, such as a spike, a ball and spike, a tamp, an elevator, a double ball spike, an awl, or the like. For simplicity in description, a ball 18 and spike 20 configuration is described herein. The ball 18 and spike 20 are configured to engage, hold and/or manipulate a fragment of bone. The ball 18 may be a part-spherical ball or spherical ball attached to or integrally formed on the elongated member 36 at the distal end 44. The spike 20 is mounted on the ball 18. Alternatively, the spike 20a may be mounted on the distal end 44 of the first tool component 14 without the ball (shown in FIG. 3B). The spike 20 or 20a can engage with a bone fragment when the surgeon manipulates the handle 12 (e.g., applies force).

While the elongated member 36 for the first tool component 14 shown in FIGS. 1 and 3A-3B is illustrated as having an arched portion 46, this particular configuration is simply one non-limiting embodiment of the elongated member 36 of the present disclosure. It will be understood that the elongated member 36 produced in accordance with the present disclosure can assume other sizes, shapes and configurations, so long as the elongated member is capable of being present in the assembly 10 disclosed herein and capable of functioning in accordance with the present disclosure. For example, but not way of limitation, FIGS. 3C and 3D illustrate several different shapes and configurations of elongated members for the first tool component 14, as described in greater detail herein below.

FIG. 3C illustrates the elongated member 36a having a cylindrical bar 50 extending from the proximal end 42a to the distal end 44a. The cylindrical bar 50 may be solid or hollow. It will be understood that the elongated member 36a is illustrated in FIG. 3C as having a cylindrical shape, however, the elongated member 36a may possess other shapes, including but not limited to, cube, prismatoid, conical, or other shape.

FIG. 3D illustrates the elongated member 36b having a curved portion 52 and a substantially straight portion 54. The curved portion 52 extends from the proximal end 42b at the angle of extension θ₁ and transitions to the straight portion 54 towards the distal end 44b of the elongated member 36b.

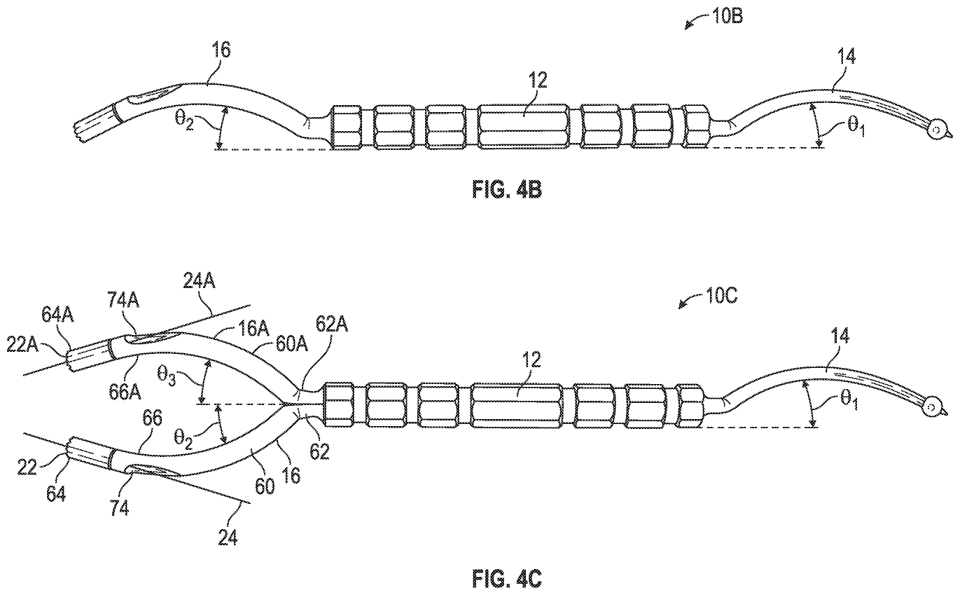

Referring to FIGS. 1 and 4A-4B, the second tool component 16 of the assembly 10 includes an elongated member 60 extending from a proximal end 62 to a distal end 64. The elongated member 60 may be formed as a hollow tube extending from the proximal end 62 to the distal end 64. A portion of the elongated member 60 may be formed as a solid rod. The elongated member 60 is shown in FIGS. 1 and 4A having arched portion 66. The arched portion 66 may extend from the handle 12 at an angle of extension θ₂ relative to the handle 12 of the assembly 10. The arched portion 66 may be similar to the arched portion 46 (shown in FIG. 3A) and may symmetrically curve from the proximal end 62 to the distal end 64 of the elongated member 60 of the second tool component 16. The angle of extension θ₂ may be configured to allow for a surgeon to apply force in a range of directions relative an incision. The angle of extension 02 may be congruent to the angle of extension θ₁ of the curved portion 46 of the elongated member 36 and serve as an alternate angle to the angle of extension θ₁ of the curved portion 46 of the elongated member 36 of the first tool component 14 as shown in FIG. 1. To that end, the angle of extension 01 and the angle of extension 02 may be congruent angles and alternate angles. In another example, the angle of extension θ₂ may be congruent and mirror the angle of extension θ₁ of the arched portion 46 of the elongated member 36 of the first tool component 14 as shown in FIG. 4B. To that end, the angle of extension 74 ₁ and the angle of extension 02 may be congruent angles and mirroring angles.

The distal end 64 of the elongated member 60 includes a tamp 22. The tamp 22 may be configured to elevate depressed areas of a fracture site, assist with stabilization of bone and bone fragments, pack bone and fragments into position, and the like. The tamp 22 illustrated in FIGS. 1 and 4A includes a serrated edge 68. The serrated edge 68 includes multiple projections 70 extending from the distal end 64 of the elongated member 60.

FIGS. 1 and 4A illustrate the tamp 22 in a cylindrical shape. The cylindrical shape is solely for purposes of illustration only and should not be considered limiting; the tamp 22 may assume any shape (including, but not limited to, cube, cuboid, conical, or frustoconical shapes, any combination of one or more shapes, etc.) so long as the shape will allow the tamp 22 to function in accordance with the present disclosure.

A cavity 72 within the elongated member 60 is configured to accept the k-wire 24. The cavity 72 may extend from the distal end 64 of the elongated member 60 to an opening 74 in an outer surface 76 of the elongated member 60. Alternatively, the cavity 72 may be configured to extend from the distal end 64 to the proximal end 62 of the elongated member 60.

The opening 74 in the outer surface 76 of the elongated member 60 may be sized and shaped to accept a k-wire 24 (shown in FIG. 1). The k-wire 24 may then extend through the cavity 72 of the elongated member and through the tamp 22 positioned at the distal end 64 of the elongated member 60. The k-wire 24 may be slidably disposed between the opening 74 in the outer surface 76 of the elongated member 60 and the tamp 22 positioned at the distal end 64 of the elongated member 60.

FIG. 4C illustrates the second tool component 16 of the assembly 10 including a third tool component 16a. The third tool component 16a includes an elongated member 60a. The elongated member 60a extends from a proximal end 62a to a distal end 64a and includes arched portion 66a. The arched portion 66a may extend at an angle of extension 74 ₃ relative to the handle 12 allowing the surgeon to apply force in a range of directions relative to an incision. The arched portions 66 and 66a may extend in opposing directions as shown in FIG. 4C. The angle of extension 74 ₃ of the arched portion 66a and the angle of extension 74 ₂ of the arched portion 66 may be similar or different.

The distal ends 64a and 64b of the elongated member 60a and the elongated member 60 may each include a tamp 22 and 22a. Alternatively, the distal end 64 and 64a of the elongated members 60 and 60a may include the tamp 22a and the distal end 64 of the elongated member 60 may be a different functional tool such as a hook, spike, impactor, and/or the like. Alternatively, the distal end 64 of the elongated member 60 may include the tamp 22 and the distal end 64a of the elongated member 60a may include a different functional tool.

Each of the elongated member 60 and 60a includes an opening 74 and 74a, respectively. Each opening 74 and 74a is sized and shaped to receive a k-wire 24 and 24a, respectively. The elongated member 60a may be cannulated between the opening 74a and the distal end 64a such that the k-wire 24a may be slidably positioned within the elongated member 60a. Similarly, the elongated member 60 may be cannulated between the opening 74 and the distal end 64 such that the k-wire 24 may be slidably positioned with the elongated member 60. To that end, the assembly 10 may include simultaneous use of two separate k-wires, with a first k-wire positioned in the opening 74a and a second k-wire positioned in the opening 74. The assembly 10 also supports the use of sequential use of the two separate k-wires, with the first k-wire positioned in the opening 74a and placed by the surgeon at the surgical site prior to positioning of the second k-wire in the opening 74.

The structure of the openings 74 and 74a, and the elongated members 60 and 60aFIGS. 4A-4C may be provided with any structural features that allow for the k-wire to be slidably disposed through or fixedly positioned within the cavity 72 and 72a.

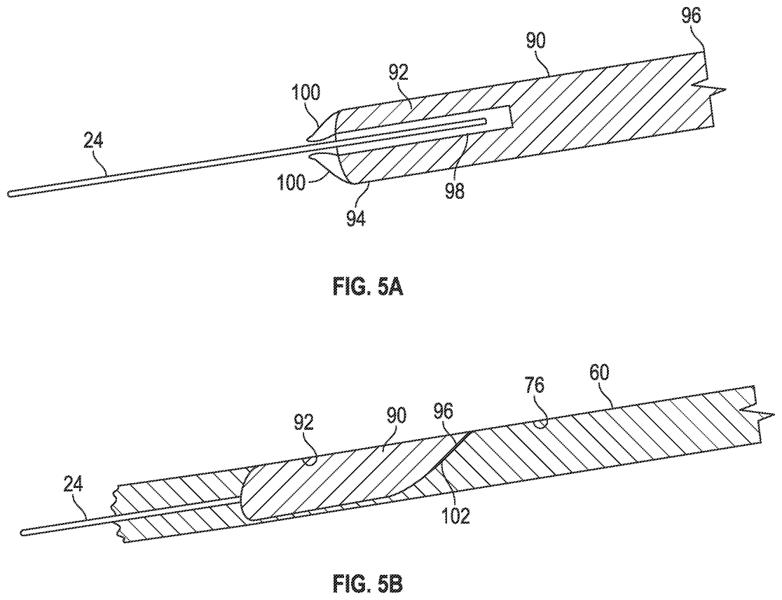

FIGS. 5A and 5B illustrate an exemplary guiding member 90 for use with the k-wire 24. The guiding member 90 is shaped and sized to be fixedly retained within the cavity 72 of the assembly 10. The guiding member 90 may guide the k-wire 24 into the cavity 72 and through the distal end 64 of the elongated member 60. Additionally, the guiding member 90 may provide support and retention of the k-wire 24 allowing the k-wire to be used to lift bone fragments during use of the assembly 10. The guiding member 90 has a sidewall 92 extending from a first end 94 to a second end 96. The sidewall 92 defines a cavity 98 extending a portion of the length of the guiding member 90 from the first end 94 to the second end 96. The cavity 98 is sized and shaped to allow for a portion of the k-wire 24 to be disposed within cavity 98. Multiple flanges 100 may be positioned at the first end 94 of the guiding member 90. The flanges 100 may be formed of spring-like material configured to be movable outwardly from a resting position to an expanded position. In the expanded position, the flanges 100 provide friction to retain the k-wire 24 within the cavity 98.

The first end 94 of the guiding member 90 is sized and shaped to be positioned in the cavity 72 of the distal end 64 of the elongated member 60. The second end 96 of the guiding member 90 is tapered to abuttingly engage with a tapered portion 102 of the elongated member 60. When the guiding member 90 is positioned within the cavity 72 of the elongated member 60, the sidewall 92 of the guiding member 90 may be flush with the outer surface 76 of the elongated member 60 as shown in FIG. 5B.

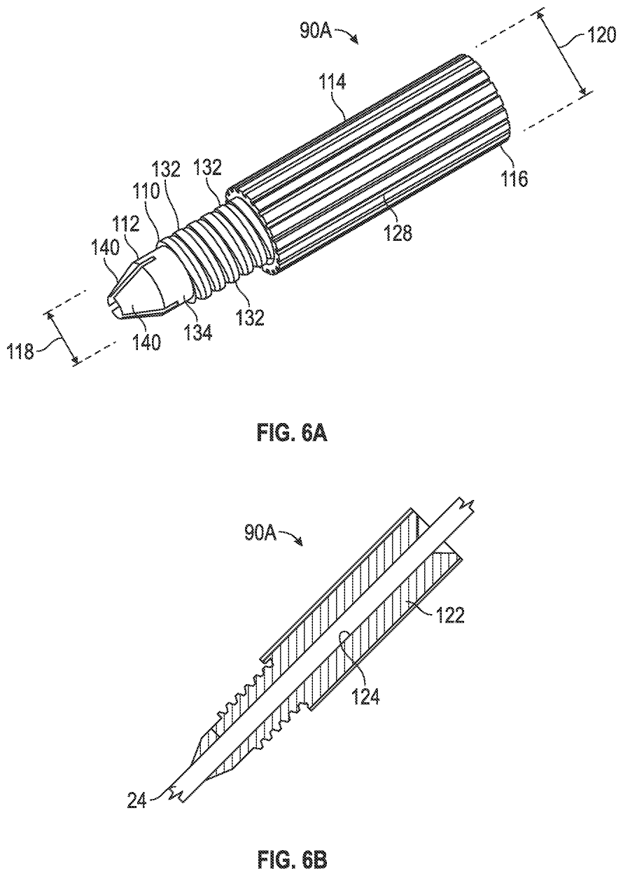

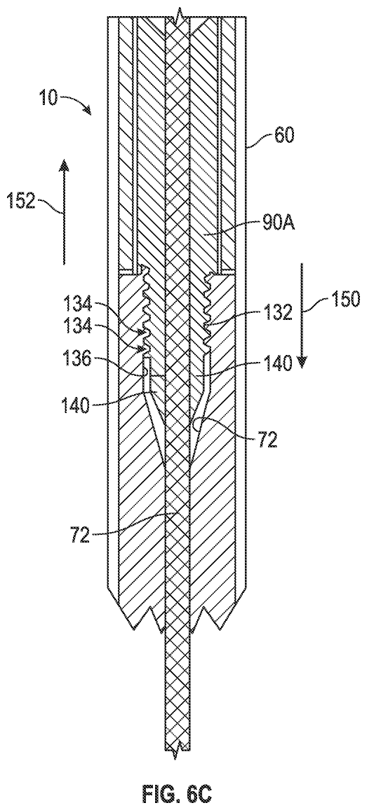

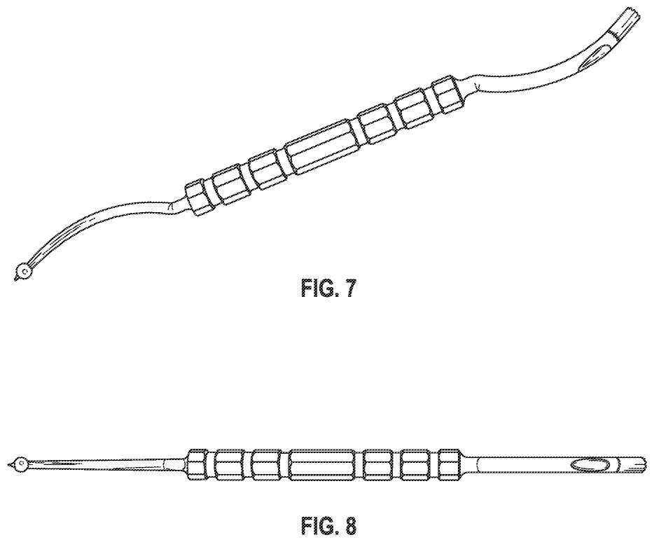

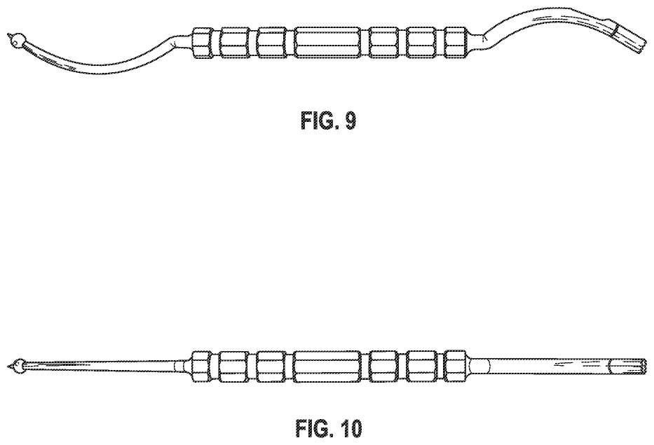

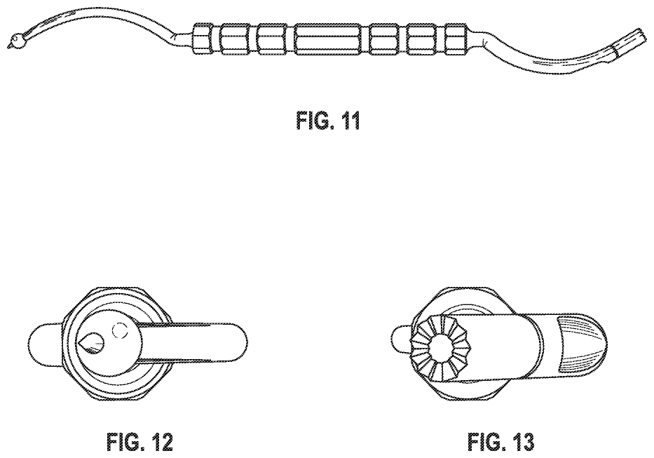

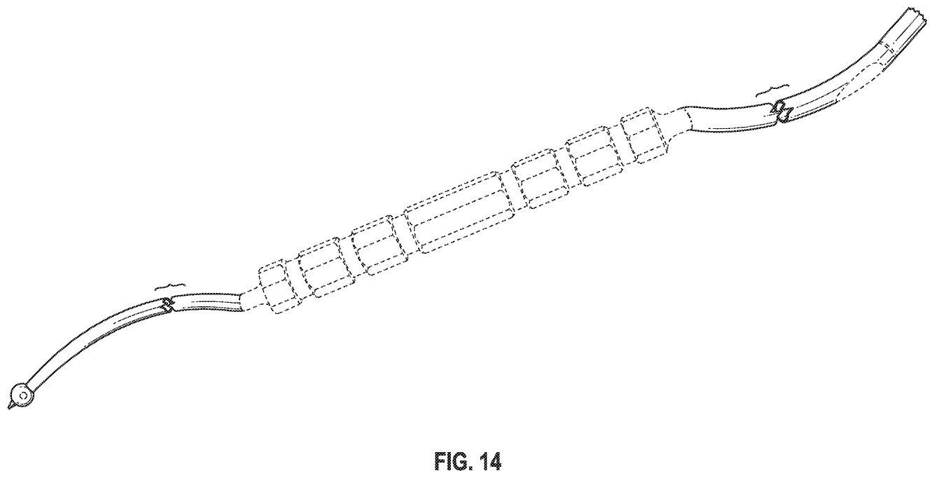

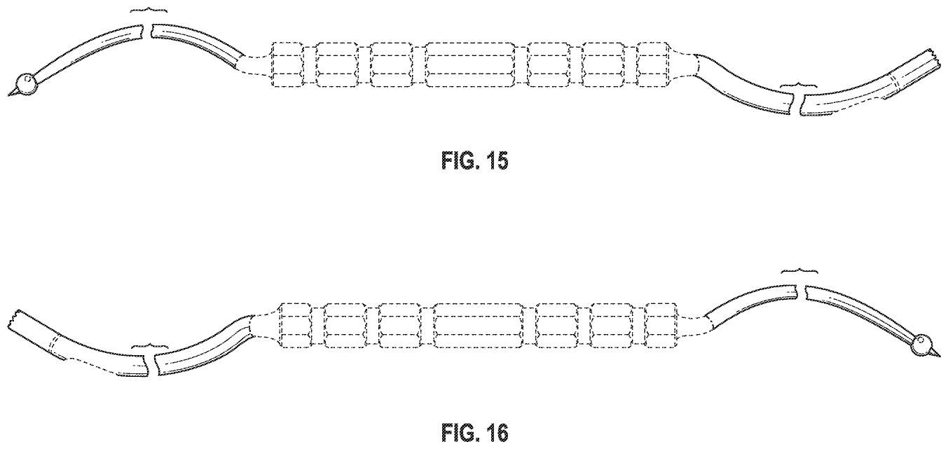

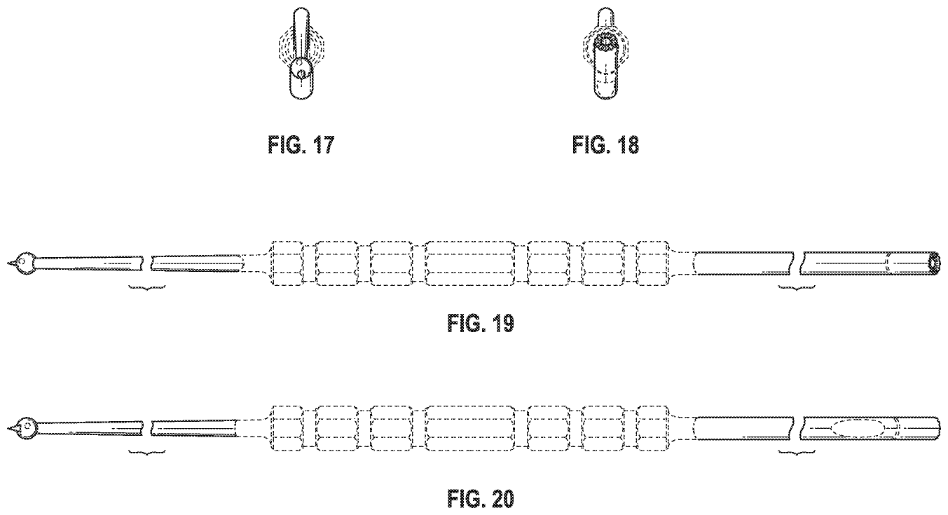

FIGS. 6A-6C illustrate another exemplary guiding member 90a for use with the k-wire 24. The guiding member 90a is shaped and sized to be fixedly retained within the cavity 72 of the assembly 10. The guiding member 90a may be used to guide the k-wire 24 into and through the elongated member 60 and may provide support and retention of the k-wire 24 allowing the k-wire 24 to be used to lift bone fragments during use.

The guiding member 90a has a connector portion 110 positioned at a first end 112 and a shaft 114 positioned at a second end 116. The connector portion 110 may have a first cross-sectional distance 118 that is less than a second cross-sectional distance 120 of the shaft 114. An inner wall 122 of the guiding member 90a defines a cavity 124 extending from the first end 112 to the second end 116 of the guiding member 90a. The cavity 124 is sized and shaped to retain the k-wire 24.

The shaft 114 includes an outer surface 126. The outer surface 126 may include one or more ribs 128 providing friction to retain the guiding member 90a within the elongated member 60. The connector portion 110 may include protrusions 132 on an exterior surface 130 of the connector portion 110 to matingly engage with corresponding recesses 134 on an inner surface 136 of the elongated member 60.

The connector portion 110 may include multiple flanges 140 positioned at the second end 116 of the guiding member 90a. The flanges 140 may be formed of spring-like material configured to be movable outwardly from a resting position to an expanded position. In the expanded position, the flanges 140 provide friction to retain the k-wire 24 within the cavity 124. Further, as the guiding member 90a is advanced through the cannulated portion of the elongated member 60 in the direction of arrow 150, the flanges 140 may be compressed against the k-wire 24 by contact of the flanges 140 with the inner surface 136 of the elongated member 60. For example, the inner surface 136 may be tapered such that the flanges 140 are compressed against the k-wire 24 by contact of the flanges 140 with the inner surface 136 as shown in FIG. 6C. The compressive force of the inner surface 136 of the elongated member 60 to the flanges 140 secures and retains the k-wire 24 within the guiding member 90a. To release the k-wire 24, the guiding member 90a is moved in the direction of arrow 152 such that the compressive force applied to the flanges 140 by the inner surface 136 of the elongated member 60 is reduced and the k-wire 24 may be released from the guiding member 90a.

Thus, in accordance with the present disclosure, there have been provided devices, assemblies, and kits, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

1. A bone fragment manipulation device for use with a k-wire, the device comprising:
a first tool component having a first elongated member extending from a proximal end to a distal end, the distal end having a spike;
a second tool component having a second elongated member extending from a proximal end to a distal end and having an outer surface defining an opening configured to receive the k-wire, the distal end of the second tool component having a tamp; and,
a handle disposed between the first tool component and the second tool component.

2. The bone fragment manipulation device of claim 1, wherein the spike is mounted on a spherical ball and configured to engage with bone fragments.

3. The bone fragment manipulation device of claim 1, wherein the first elongated member includes a first arched portion extending from the handle at a first angle and symmetrically curving from the proximal end to the distal end of the first elongated member.

4. The bone fragment manipulation device of claim 3, wherein the second elongated member includes a second arched portion extending from the handle at a second angle and symmetrically curving from the proximal end to the distal end of the second elongated member.

5. The bone fragment manipulation device of claim 4, wherein the first angle and the second angle are approximately congruent angles.

6. The bone fragment manipulation device of claim 5, wherein the first angle and the second angle are approximately alternate angles.

7. The bone fragment manipulation device of claim 1, wherein the tamp includes a serrated edge having multiple projections extending from the distal end of the second elongated member.

8. The bone fragment manipulation device of claim 1, wherein the second elongated member includes a cavity extending from the opening to the distal end of the second elongated member and configured to receive the k-wire slidably disposed within the cavity from the opening and through the tamp at the distal end of the second elongated member.

9. The bone fragment manipulation device of claim 8, further comprising a guiding member configured to be fixedly retained within the cavity of the second elongated member, the guiding member having a sidewall extending from a first end to a second end and defining a first cavity configured to retain the k-wire.

10. The bone fragment manipulation device of claim 9, wherein the guiding member includes a plurality of flanges positioned on the second end, the flanges configured to be movable in an expanded position to retain the k-wire within the first cavity.

11. The bone fragment manipulation device of claim 9, the guiding member having a connector portion and a shaft, the connector portion including a plurality of protrusions on an exterior surface of the connector portion configured to engage with recesses on an inner surface of the second elongated member.

12. The bone fragment manipulation device of claim 1, further comprising a third tool component having a third elongated member extending from the handle and positioned adjacent to the second tool component.

13. The bone fragment manipulation device of claim 12, wherein the third elongated member extends from a proximal end to a distal end and has an outer surface defining an opening configured to receive a second k-wire, the distal end of the third tool component having a second tamp.

14. A kit, comprising:
the bone fragment manipulation device of claim 1; and
at least one k-wire.

15. The kit of claim 14, further comprising a guiding member configured to be fixedly retained within a cavity of the second elongated member extending from the opening to the distal end of the second elongated member, the guiding member having a sidewall extending from a first end to a second end and defining a first cavity configured to retain the k-wire.

16. The kit of claim 15, wherein the guiding member includes a plurality of flanges positioned on the second end, the flanges configured to be movable in an expanded position to retain the k-wire within the first cavity.

17. A method of manipulating bone fragments about a surgical site of a patient, the method comprising the steps of:
(1) inserting a bone fragment manipulation device in an incision in skin of the patient and directing the bone fragment manipulation device to the surgical site, the bone fragment manipulation device having a handle disposed between a first tool component and a second tool component;
(2) positioning the first tool component of the bone fragment manipulation device at the surgical site;
(3) applying force via the handle to the first tool component, the first tool component having a spike mounted on a ball, wherein the force applied to the handle allows the spike to manipulate bone fragments at the surgical site;
(4) rotating the handle of the bone fragment manipulation device such that the second tool component is positioned at the surgical site;
(5) applying force via the handle to the second tool component, the second tool component having tamp, wherein force applied to the handle allows the tamp to hold bone fragments at the surgical site; and,
(6) inserting a k-wire into an opening in the second tool component.

18. The method of claim 17, further comprising a step of applying force to the handle to manipulate bone fragments via the k-wire.

19. The method of claim 17, further comprising a step of inserting a second k-wire into an opening of a third tool component of the bone fragment manipulation device and applying force to the handle to manipulate bone fragments via the second k-wire.

20. The method of claim 17, wherein applying force via the handle to the second tool component elevates die punch fragments.
